(19) United States
(12) Patent Application Publication
(10) Pub. No.: US 2004/0100616 A1
(43) Pub. Date: May 27, 2004

(54) Method and device for spectral optical reflector therapy for improving vision (variants)

(76) Inventor: Alexandr Pavlovich Eremeev, Moscow (RU)

Correspondence Address: DELLETT AND WALTERS, P. O. BOX 2786, PORTLAND, OR, 97208-2786, US

(21) Appl. No.: 10/250,652

(22) PCT Filed: Nov. 26, 2001
(86) PCT No.: PCT/RU01/00508

(30) Foreign Application Priority Data
Jan. 5, 2001 (RU) 2001100289

(51) Int. Cl. A61B 3/00

(52) U.S. Cl. 351/203

(57) ABSTRACT

The invention relates to medicine. In the first variant, the inventive method consists in a cyclic modification of a degree of tension for eye accommodation. Said modification is produced by changing the frequency content of the more intense light flux. One flux forms images of visual stimulus on the retina, the other flux forming colour backgrounds thereof. The frequency content is modified using a cyclic magnification and reduction of a wavelength value of a location of the spectral distribution centre of the more intensive light flux. In the second variant, cyclic modification of the intensity ratio of the light fluxes is carried out. One flux forms images of a visual stimulus on the retina, the other flux forms the colour backgrounds thereof, said fluxes being embodied in such a way that they alternately become more intensive. The inventive device comprises a unit embodied in such a way that the light-luminance characteristics of the visual stimulus and the colour backgrounds thereof are modifiable. Said unit makes it possible to carry out a cyclic modification of the light-luminance characteristics of the more luminous visual stimulus and the colour backgrounds thereof with respect to each other.

### FIELD OF INVENTION

This invention relates to the field of medicine, and, more particularly, to ophthalmology, and may be used for improving the visual functions.

### PRIOR ART

One way of improving vision or preventing its disorders is retinal work in the conditions of purposeful modifications in the degree of the eye tension accommodation. For those purposes various methods and devices are used: optical loads with the use of optical elements (lenses)—the Avetisov-Mats method, retinal work with the table of optotypes of different sizes with the use of optical elements (lenses)— the “build-up” method.

The method of improving vision (“build-up” method) comprises modifications (increase and decrease) in the degree of the eye tension accommodation by presenting optical elements—lenses (1).

The procedure of accommodation training by the “build-up” method consists in the following.

The patient is seated at a distance of 5 meters from the table for determining the visual acuity. Into a test frame before each eye spherical glasses are successively placed, starting from +0.75 diopter. The patient is invited to look at the table attentively. At first, with the +0.75 diopter glass the patient can see the table poorly, but in 3 to 5 minutes the visual acuity increases. The glasses are replaced with spherical −0.75 diopter ones, which are left in the frame for only 20 to 30 seconds. Due to a rapid accommodation change the patient can distinctly see the 10th line of the table. Then into the same frame spherical +0.5 diopter glasses are placed (removing −0.75 diopter ones) for 2 to 3 minutes. Those glasses are replaced with −0.5 diopter ones (for 15 to 20 seconds). Then the procedure is repeated with +0.25 and −0.25 diopter lenses.

The shortcomings of the known methods are: the complexity of procedures for improving of vision, which is associated with the use of a special set of lenses; the use of optotypes tables, which do not ensure psychoemotional stimuli; the difficulty of broad usage outside medical institutions.

Also known in the art is a device for improving the eye resolution (2). The device comprises color filters and the body made with the possibility of their installation and modification before a carrier, on which visual stimuli are made. The color filters are made in different colors and are intended for modifying the color background of the visual stimuli.

When using that device, a patient is invited, at a distance of 5 meters, to look at the table arranged in the device and containing visual stimuli—test figures corresponding, as to the angular dimensions and the space-frequency spectrum, to optotypes in the Golovin-Sivtsev table for testing visual acuity. A patient, by using a device for brightness adjustment, selects a level of illumination, at which the test figures on the table are most distinct. Then the patient, having concentrated attention on a table line corresponding to his/her visual threshold, cyclically and continuously adjusts the illumination level of a part of the table, which is the color background of the test figures, while intending to preserve the distinct vision of the figures in the range of adjusted illumination. In 5 to 7 minutes the patient places a changeable color (red, green, blue or yellow) filter and repeats training in the conditions of presenting test figures on a color background again for 5 to 7 minutes. The influence with other replaceable color filters is made similarly.

The shortcomings of the said devices are: they stimulate the retina neuroreceptors only; optotypes tables are used, which do not ensure psychoemotional stimulation; there are no criteria for selecting the parameters of filter colors and for order of their replacement. Furthermore, only one-color filters are used in that technical solution. The device is intended for use in clinical conditions due to its big dimensions and complex construction.

However, the known in the art methods and devices do not make spectral optical reflex effects on the visual system.

### DISCLOSURE OF THE INVENTION

This inventions is based on the objective of creating a method of spectral optical reflex therapy, which would simplify the procedures for improving vision and the equipment applied therefore and which may be broadly used outside medical institutions, as well as of creating devices for realizing the said method, having expanded functional possibilities for implementing new procedures for improving vision and simplified constructions, thus ensuring the improvement of quality and enlargement of the range of means for improving the functions of the visual system.

For achieving the stated objective the first variant of the claimed method of spectral optical reflex therapy for improving vision includes cyclic adjustment of the degree of the eye accommodation tension, which is done by changing the spectral distribution of the more intensive light flux out of the available ones, one of which forms images of visual stimuli on the retina and the other one forms color backgrounds of the visual stimuli, while the spectral distribution is changed by cyclically increasing and decreasing the wavelength of the position of the spectral intensity distribution center of the more intensive light flux out of the said ones.

Additional embodiments of the method according to the first variant are possible, wherein it is advisable that:

the spectral distribution would be changed by cyclically increasing and decreasing the wavelength of the spectral intensity distribution by the maximum value of, at least, 3 nanometers;

the spectral distribution of the more intensive light flux out of the said ones would be changed with a frequency not more than 4 Hz.

For achieving the stated objective the second variant of the claimed method of spectral optical reflex therapy for improving vision includes cyclic adjustment of the degree of the eye accommodation tension, which is done by cyclically modifying the intensity relation of the light fluxes, one of which forms images of visual stimuli on the retina and the other one forms color backgrounds of the visual stimuli by alternatively making one of the light fluxes more intensive, while the light fluxes are formed with the spectral distributions having different wavelengths of the positions of the spectral intensity distribution centers before and after modifying the intensity relation of the light fluxes.

Additional embodiments of the method according to the second variant are possible, wherein it is advisable that:

the wavelengths of the positions of the spectral intensity distributions centers of the light fluxes before and after modifying their intensity relation would be selected with the difference of, at least, 3 nanometers;

the relation of the light flux intensities would be changed with a frequency of not more than 4 Hz.

For achieving the stated objective with the yield of the stated technical result the first variant of the claimed device for spectral optical reflex therapy for improving vision comprises a means made with the possibility of cyclically modifying the color parameters of visual stimuli, which would be brighter than their color backgrounds, while the wavelengths of the positions of the spectral intensity distribution centers of the light fluxes, which are formed by brighter visual stimuli before and after modifying their color parameters, are different.

In addition to the first main variant of making the device, an additional variant is possible, wherein it is advisable that:

the said means would be made with the possibility of modifying color-brightness characteristics of the visual stimuli and their color backgrounds made by light reflecting means of presenting visual information.

In addition to the above additional variant, some variants are possible, wherein:

the said means would be made as a carrier, on which visual stimuli that are different as to their color parameters would be arranged;

the said means would be made in the form of a carrier and a transparent light filter which is arranged before the carrier and has visual stimuli with different color parameters arranged on it;

the said means would be made as a projection screen;

the said means would be made as a carrier, on which visual stimuli are arranged, and a transparent color filter arranged before it.

In addition to the above additional variant, a variant is possible, wherein:

a transparent color filter would consist of parts having different color parameters.

For achieving the stated objective with the yield of the stated technical result the second variant of the claimed device for spectral optical reflex therapy for improving vision comprises a means made with the possibility of cyclically modifying the color parameters of the color backgrounds, which are brighter than the visual stimuli made thereon, wherein the wavelengths of the positions of the spectral intensity distribution centers of the light fluxes, which are formed by the brighter color backgrounds before and after modifying their color parameters, are different.

In addition to the second main variant of making the device, an additional variant is possible, wherein it is advisable that:

the said means would be made with the possibility of modifying the color-brightness characteristics of the visual stimuli and their color backgrounds, which are made by light reflecting means for presenting visual information.

In addition to the above additional variant, some variants are possible, wherein:

the said means would be made as a carrier, on which visual stimuli that are different as to their color parameters would be arranged;

the said means would be made in the form of a carrier and a transparent light filter which is arranged before the carrier and has visual stimuli with different color parameters arranged on it;

the said means would be made as a projection screen;

the said means would be made as a carrier, on which visual stimuli are arranged, and a transparent color filter arranged before it.

In addition to the above additional variant, a variant is possible, wherein:

a transparent color filter would consist of parts having different color parameters.

For achieving the stated objective with the yield of the stated technical result the third variant of the claimed device for spectral optical reflex therapy for improving vision comprises a means made with the possibility of cyclically modifying the color parameters of color backgrounds, which would be brighter than the visual stimuli made thereon, while the wavelengths of the positions of the spectral intensity distribution centers of the light fluxes, which are formed by brighter color backgrounds before and after modifying their color parameters, are different.

In addition to the third main variant of making the device, an additional variant is possible, wherein it is advisable that:

the said means would be made with the possibility of modifying color-brightness characteristics of the visual stimuli and their color backgrounds made by light reflecting means for presenting visual information.

In addition to the above additional variant, some variants are possible, wherein:

the said means would be made as a carrier with the parts different as to their color-brightness characteristics, the said parts being the color backgrounds of the visual stimuli arranged on the carrier and made with different color-brightness characteristics;

the said means would be made in the form of a carrier with the parts different as to their color-brightness characteristics and a transparent light filter which is arranged before the carrier, on which the visual stimuli are arranged, the said visual stimuli being made non-transparent and different as to their color-brightness characteristics;

the said means would be made as a projection screen.

In addition to the first, second and third main variants of making the device, an additional variant is possible, wherein it is advisable that:

the said means would be made with the possibility of modifying color-brightness characteristics of the visual stimuli and their color backgrounds made by light reflecting means for presenting visual information.

In addition to the above additional variant, some variants are possible, wherein:

the said means would be made as a computer display;

the said means would be made as a TV-set screen.

The above methods and device do not ensure spectral optical reflex effects on the visual system and do not enable to conduct mass actions for improving vision, since they require the participation of medical specialists, carrying-out of tiresome special trainings in the absence of psychoemotional stimulation, as well as the use of ophthalmologic equipment. Therefore, it has been necessary to develop non-traditional and original methods and devices, which would ensure the health-improving effect in the process of the usual visual work performed by a human being (reading, writing, visual perception of the outward things).

Every object of the outward things, which is available for visual perception, is a light source. Two main types of light sources are distinguished: the active sources irradiating the light, and the secondary sources, reflecting the light. These are characterized by the brightness and the spectral distribution of the light irradiated or reflected by them. It is the totalities of such light sources that form color-brightness compositions consisting of visual stimuli, which the eye is capable of accommodating on, and their color backgrounds perceived and interpreted by the visual system when perceiving the outward things.

The succession of visual stimuli, on which the eye accommodates in the process of performing the visual work, may be made, e.g., as a set of invariable stimuli, which are successively accommodated on when changing the vision direction. The examples this are books, pictures, non-dynamic images on computer displays. It is also possible to make visual stimuli in the dynamic mode—these are time-changing computer or television images.

An image on the eye retina is formed by the light flux reflected, transmitted or irradiated by objects of the outward things, which may be the objects of visual fixation where each object may be conceived as two parts, namely: a visual stimulus and its color background. A part of that light flux may be subdivided into two fluxes. One light flux forms an image of the visual stimulus on the retina, and the other flux forms the color background of the visual stimulus. Any of those fluxes is characterized by its intensity and spectral distribution. Intensities and spectral distributions of those light fluxes depend on the optical characteristics (color and brightness) of the visually fixed objects—the visual stimuli and their color backgrounds. A change in the color-brightness characteristics of parts of those objects corresponds to a change in the parameters of those light fluxes.

The spectral distribution of any light flux may be described by the function of distribution of its spectral component intensities. One of the characteristics of this function is the wavelength λ ct of the position of the spectral distribution intensity center of the light flux.

$$λ c t = \frac{{\int }_{{λ}_{v}}^{{λ}_{k}} {Φ}_{λ} \cdot λ \cdot ⅆ λ}{{\int }_{{λ}_{v}}^{{λ}_{k}} {Φ}_{λ} \cdot ⅆ λ} ,$$

where

λ—wavelength of a light wave in the visible electromagnetic wave band;

λᵥ—shortwave limit of light wavelengths in the visible electromagnetic wave band;

λₖ—longwave limit of light wavelengths in the visible electromagnetic wave band;

Φ_{λ}—function of spectral distribution of the light flux intensities.

A visual stimulus displays a color and brightness contrast in relation to the color background. In the real conditions the brightness of the stimulus and that of its background are different. When the brightness of the stimulus and that of its background are equal (in different conditions there exist insignificant differences in perceptions when comparing the levels of the brightness of the stimulus and that of its background), the possibility of recognizing the stimulus is determined by their color contrast.

In the process of visual work the optical set-up of the eye is changed in such a way so as to ensure the sharpest vision of a visual stimulus (made brighter than its color background), on which the eye accommodates in a given moment of time. The accommodation is stimulated by a loss in the sharpness of vision in the area where the visual stimuli are located, due to non-adequate convergence of light beams on the retina. The criterion for assessing the image definition on the eye retina is the maximum frequency of pulses emanating from an individual ganglious cell, on the receptors of which the light flux, which forms the image of a visual stimulus, falls. That frequency is determined by the visual stimulus brightness, i.e., by the density of photon distribution within the limits of the summation area of the given ganglious cell. In a case where the brightness of a stimulus is less than that of its background, all the above-said is correct in relation to the color background of the visual stimulus.

The refraction coefficient of optical media (including that of an eye) differs for light beams of different wavelength. The posterior focal length for beams of the shortwave (blue) range of the visible electromagnetic wave band is shorter than for beams of the long-wave (red) range. At equal other conditions the value of the posterior focal length for one-color, most short beams of the visible electromagnetic wave band is minimal, and for most long ones—maximal. At one and the same refracting power of the eye the difference between the said values may amount to 1.5 diopters. Thus, the degree of accommodation tension, which is necessary for focusing long-wave and shortwave light fluxes on the retina, will differ. In the real conditions, a light flux consists of beams having different wavelengths, and its spectral distribution may be characterized, inter alia, by the wavelength λ ct of the position of the spectral distribution center of light flux intensities, which a definite value of the posterior focal length of the eye and, consequently, a definite degree of accommodation tension, correspond to.

As studies show, a change in the degree of accommodation tension occurs at changes in the spectral distribution, at which the wavelength λ ct is changed. Thus, changes in the color characteristics of visual stimuli and their color backgrounds, when the eye performs visual work, result in changes in the spectral distribution of the light flux coming into the eye, which, in its turn, is accompanied by a change in the accommodation tension degree.

The focusing of the eye optical system is carried out with due regard to the spectral characteristics of the more intensive (creating higher illumination of the retina) light flux out of the two ones forming the image of the stimulus and its color background. And the characteristics of those light fluxes depend, respectively, on the optical characteristics of visual stimuli and their backgrounds—their brightness and color parameters—presented to the eye for perception. The modem technical level of spectrophotometry enables to define the characteristics of such light fluxes rapidly and with a high accuracy.

When the center of intensity spectral distribution of the more intensive light flux moves to the range of shorter or longer waves of the visible electromagnetic band, which may be ensured by modifying color-brightness characteristics (colors and/or brightness) of visual stimuli and their color backgrounds, the optical set-up of the eye will change and will not correspond to the optimal focus of an image on the eye retina, i.e., the definition of the visual stimulus vision will become worse. For restoring the highest possible vision definition the accommodation tension degree will change correspondingly (will increase or decrease). Therefore, a modification in the spectral distribution of the more intensive light flux forming the image of a visual stimulus or its color background is accompanied by a corresponding change in the degree of accommodation tension. Cyclic modifications in colors and brightness of visual stimuli and/or their color backgrounds, as studies show, may be used for the purposeful influence on the eye accommodation apparatus instead of the optical light-refracting elements (lenses or prisms) that are traditionally used.

For modifying the accommodation tension degree of the eye it is sufficient to effect a change in the color-brightness characteristics (change of colors and/or brightness) of a visual stimulus and/or its background. Also changed will be the relation of accommodative convergence to accommodation (AC/A), the degree of contrast of the background and the visual stimulus, and changes in the intensity of the light flux coming into the eye will entail changes in the pupil size. Thus, cyclic modifications of the color of a visual stimulus and/or its background will contribute not only to the stimulation of the accommodation apparatus of the eye and the normalization of the convergence-accommodation relations, but also to an increase of the contrast sensitivity of the eye, velocities of the accommodation reactions, stability of clear vision and stimulation of the muscle apparatus of the sphincter muscle of the pupil.

The principle of the claimed, in this invention, spectral optical reflex influence on the visual system for the purpose of improving vision in the process of performing visual work by the myopic eye is cyclic modifications in the spectral distribution of a light flux determining the degree of the eye accommodation tension. The said influence results in a movement of the wavelength of the position of the spectral distribution center of light flux intensities to the range of shorter waves of the electromagnetic wave visible band, being accompanied by worsening the vision definition of visual stimuli, and a back movement to the range of longer waves. For a hypermetropic eye the principle is cyclic modifications in the spectral distribution of a light flux determining the degree of the eye accommodation tension, leading to a movement of the wavelength of the position of the spectral distribution center of light flux intensities to the range of longer waves of the electromagnetic wave visible band, being accompanied by worsening the vision definition of visual stimuli, and a back movement to the range of shorter waves. This enables to conduct both preventive measures and treatment of refractive disorders of vision.

The peculiarity of making and functioning of the claimed, in this invention, device of optical reflex therapy is the presentation of visual information in the form of a succession of visual stimuli and their color backgrounds, as made with different, cyclically modified color-brightness characteristics, which presentation ensures that the degree of eye accommodation tension changes in accordance with the principle of the influence, as claimed in this invention, on the visual system for the purpose of improving vision in the process of visual work.

The degree of accommodation tension may be modified both due to cyclically changed, e.g., by software means on computer display, color parameters or brightness value relationships of visual stimuli and their color backgrounds and due to the alternate fixation of sight on various visual stimuli made, for example, by a method of printing, in various combinations of color parameters and brightness values of visual stimuli and their color backgrounds.

The spectral optical reflex therapy, fundamentals of which are for the first time proposed in the claimed variants of the vision-improving method and which is conducted with the use of variants of the claimed device, is notable for its efficacy, simplicity and a wide range of technical solutions for its application.

The said advantages as well as the specific features of this invention are explained with its preferred embodiments with references to the drawings.

### BRIEF DESCRIPTION OF THE DRAWINGS

FIG. 1 shows a part of an image formed on the eye retina with the use of any of the claimed devices, one of variants;

FIG. 2 is the same as FIG. 1, another variant;

FIG. 3 is the same as FIG. 1, another variant;

FIG. 4 is the same as FIG. 1, another variant;

FIG. 5 is the same as FIG. 1, another variant;

FIG. 6 is the same as FIG. 1, another variant;

FIG. 7 is the same as FIG. 1, another variant;

FIG. 8 is the same as FIG. 1, another variant;

FIG. 9 represents a color filter with the gradient (continuous) coloration;

FIG. 10 represents a color filter with the discrete (contrast) coloration.

All the Figures are made in black-and-white. In FIGS. 1-10 white, gray and black colors show changes in color and brightness values of visual stimuli 1 and their color backgrounds 2.

Since the claimed method is realized in the functioning of the device, the description of the method variants is given in describing the functioning of the device variants.

### PREFERRED EMBODIMENTS

A device for spectral optical reflex therapy for improving vision, which forms images (FIGS. 1-8) on the retina, comprises a means intended for modifying the degree of eye accommodation tension and made with the possibility of modifying the color-brightness characteristics of the visual stimuli 1 and their color backgrounds 2. In different variants of the device the said means is made so as to ensure cyclic modifications of the color parameters of the visual stimuli 1, which are brighter than their color backgrounds 2, or those of the color backgrounds 2, which are brighter than the visual stimuli 1 made thereon. A specific feature of the devices is that the center position wavelengths of spectral distribution of intensities of the light fluxes, which are formed by the brighter visual stimuli 1 or the brighter color backgrounds 2 before and after modifying their color parameters, differ. In another variant of the device the said means is made so as to ensure cyclic modifications of the brightness relations of the visual stimuli 1 and their color backgrounds 2, when the visual stimuli 1 and their color backgrounds 2 are alternatively made brighter in relation to each other. In this variant the color parameters of the brighter visual stimuli 1 or their color backgrounds 2 before and after modifying the brightness relationship are made different, and the center position wavelengths of spectral distribution of intensities of the light fluxes, which are formed by the brighter visual stimuli 1 or the brighter color backgrounds 2 before and after modifying their color parameters, are also made different.

The device for spectral optical reflex therapy for improving vision may be made with the use of various means.

For example, all variants of the device for spectral optical reflex therapy for improving vision may be made on the basis of light-irradiating means for presentation of visual information, e.g., a computer display or a television screen. Such a device has a screen, on which visual stimuli 1 are arranged that are reproduced on it in the form of, e.g., pictures and/or text matter on a color background 2 (FIGS. 1-4). The hardware cyclic modifications of colors and brightness values of the visual stimulus 1 and its color background 2 (provided by known technical means, e.g., Microsoft Word 97, or by playing video records) enables to conduct measures preventing vision disorders or improving visual functions in domestic conditions.

Moreover, variants of the device may be made on the basis of light-reflecting means for presentation of visual information. For example, the visual stimuli 1 and their color backgrounds 2 may be made in the form of slides or transparency films, which are projected onto a projection screen (FIGS. 1-8).

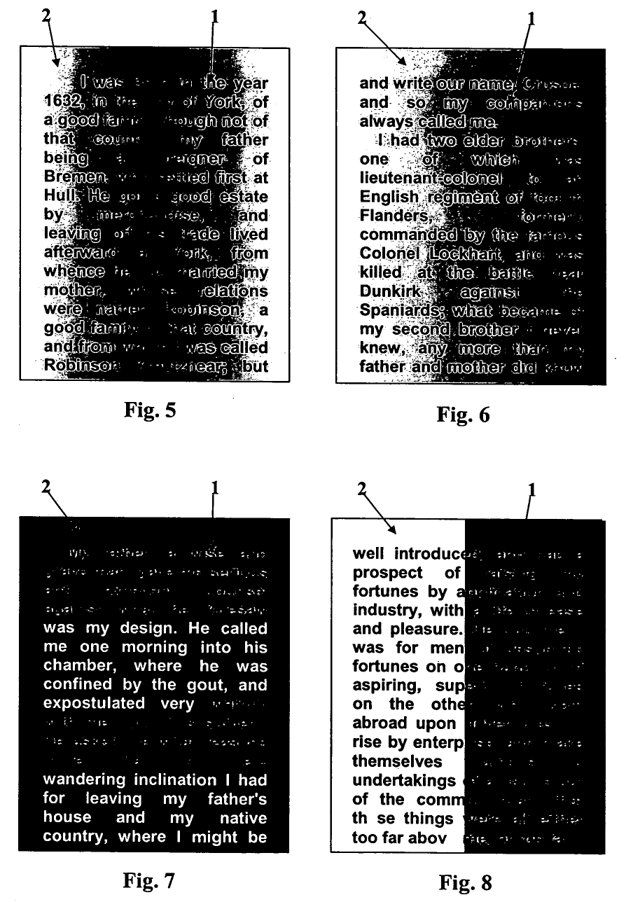

Also, the device for spectral optical reflex therapy for improving vision may comprise a carrier (made of paper, plastics, cloth, photo materials or any other material) with the possibility of arranging (being glued, on magnetic basis or by any other method) or making (by printing, photography, drawing, optically projecting or by any other method) visual stimuli 1 and their color backgrounds 2 (FIGS. 5-8) thereon. Thus, the visual stimuli 1 and parts of the carrier, which represent the color backgrounds 2, may be made transparent and/or opaque, with different color parameters and/or brightness values. A modification of the spectral distribution of brighter light fluxes, which form images of the visual stimuli 1 and their color backgrounds 2 on the retina, may be effected by successive visual fixation on various visual stimuli 1 (FIGS. 5-8).

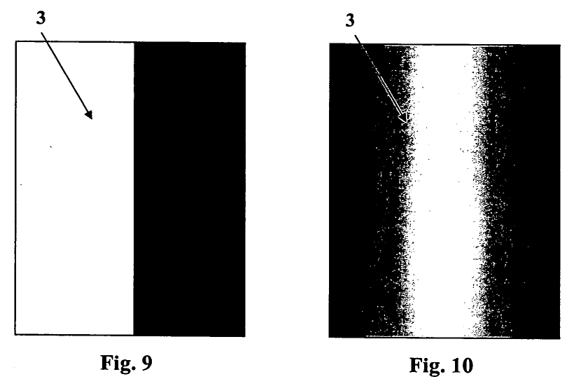

Also, various combinations of the carrier and a transparent color filter 3, as arranged before the carrier, may be used. The visual stimuli 1 and their color backgrounds 2 may be made separately, with different color-brightness characteristics, on the carrier and on the color filter 3. For example, the color filter 3 may be made with discrete (contrast) (FIG. 9) and/or gradient (gradual, continuous) (FIG. 10) change of the color parameters and may be arranged before the carrier with its visual stimuli (not shown in FIGS. 9, 10), fully or partially overlapping the carrier. Differently colored parts of the color filter 3 are shown in FIGS. 9, 10 by white and gray colors. Liquid-crystal color filters may be used as color filters 3.

Also, the devices for spectral optical reflex therapy for improving vision may be made as additional light sources with variable or constant spectral distribution of the irradiated light flux for illuminating the carrier with visual stimuli 1 made thereon.

A device for spectral optical reflex therapy for improving vision may be made in the form of a helmet, or a ring, or spectacles, which may be conveniently fixed on the head. In the body of such a device color filters 3 are installed, which may be made, e.g., in one color, be arranged before the eyes and may fully or partially overlap the field of eyes' vision. Color filters 3 may be replaced manually, for what they may be made, e.g., in the form of a plate moving in the body's slots or in the form of sectors on a disk being rotated before the eyes. Also, used may be color filters 3 made discretely (FIG. 9) or with gradient (FIG. 10) as to their color tone and depth and overlapping the vision fields of the eyes. A modification of the spectral distributions of light fluxes inducing color sense of the spectral and brightness composition of the visual stimuli 1 and their color backgrounds 2, as projected onto the retina, is effected by changing the sight direction and fixing the visual stimuli 1 through differently colored parts of the color filter 3 (FIGS. 5, 6, 8). The color filters 3 may be made as optical elements, i.e., lenses, prisms. This expands the possibilities of using the device depending on the nature of a vision disorder.

Other different variants of the device are also possible, which will be evident for specialists in the art from the essence of the claimed technical solution and which enable to achieve the stated objective with the use of the means described. But in any variants of technical making of the device the condition of cyclically decreasing and increasing the center position wavelengths of spectral distribution of intensities of the light fluxes, which are formed by the brighter visual stimuli or the brighter color backgrounds, should be complied with at successive accommodation of the eye on the visual stimuli 1 (FIGS. 1-8).

The influence on the visual system in accordance with variants of the claimed method and with the functioning of the variants of the device is effected as follows.

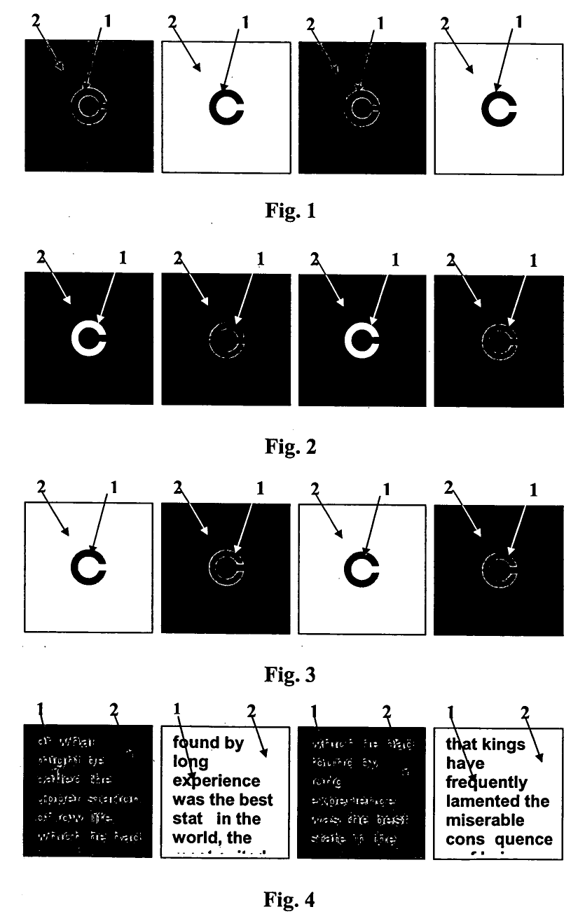

At first, the minimal visual stimuli 1, which correspond, as to their angular dimensions, to the maximum acuity of vision, are presented before the eye at the initial distance and at a given initial composition of colors and brightness of the visual stimuli 1 and their color backgrounds 2. In some time, the definition of perception, which is individual for each eye and depending on the speed of accommodation reactions and accommodation resources, is restored. At this, the degree of accommodation tension is changed, and the optical set-up of the eye will correspond to clear vision of the visual stimuli 1 already having lesser angular dimensions at the initial spectral composition of the visual stimuli 1 and their color backgrounds 2. After the clear visual perception is established, the color-brightness characteristics of the visual stimuli 1 and/or their color backgrounds 2 are modified so as to worsen the clearness of perception of the presented visual stimuli 1. For example, in the beginning of the influence a myopic eye accommodates on the red (made in white color in FIG. 2a) visual stimulus 1 made against the black background. Then the color of the visual stimulus 1 is changed for blue (made in gray color in FIG. 2b). After the initial vision definition of the visual stimulus 1 is restored, the color of the visual stimulus 1 is changed for red, and the angular dimensions are decreased (e.g., by 3 to 10 percent compared to the initial ones). If the initial vision definition is not restored, the angular dimensions of the stimuli are not changed (FIG. 2c). Then the influence is repeated. Later on, the visual stimuli 1 of lesser dimensions, which correspond to the established higher vision acuity, are presented. This cycling is shown on FIG. 2d.

FIG. 7 presents a variant in accordance with the claimed method, which realizes modifications of the brightness values and the color parameters of the visual stimuli 1 without modifying the color-brightness characteristics of the color background 2. The color backgrounds 2 may be modified similarly, while preserving the color-brightness characteristics of the visual stimuli 1 (FIG. 8). The visual stimuli 1 may be printed in blue paper in different colors, e.g., as black and red letters of the text matter (FIG. 7). Red letters are made brighter in relation to blue paper (shown in white and gray colors in FIG. 7). In the process of influence the sight is successively fixed on various visual stimuli 1 (reading of text), thus effecting stimulation.

For the purposes of preventing vision disorders the color parameters of the visual stimuli 1 and/or their color backgrounds 2 may be alternatively modified toward shorter-wave and longer-wave ranges of the electromagnetic wave visible band. The influence is effected as follows. The visual stimuli 1 are presented to the eye alternatively against the initial color background 2, then the color of the background 2 is changed toward short or long waves, then it is returned to the initial one, and then the color of the background 2 is inversed from the effected change. This cycle is repeated. For example, reading of text made in black letters on the carrier of different colors (FIGS. 5, 6, 8). Reading of a book through replaceable, e.g., red and blue color filters 3 is also possible, when each next page may be read through another color filter 3. In another variant the text page is only partially covered with transparent color filter 3. Reading of a page in parts through different color filters 3 or through color filters 3 with differently colored parts (FIGS. 9, 10) is also possible.

In the process of influence the wavelength of the position of the center of intensity spectral distribution of the more intensive light flux is cyclically increased and decreased by a maximum value of not less than 3 nanometers, since, as practical studies show, the degree of eye accommodation tension is not changed at lesser values.

It is advisable that the frequency of modifying the color parameters and/or brightness relations of the visual stimuli 1 and their color background 2 should not exceed 4 Hz due to the fact that a mean time of accommodation reaction of a healthy eye is more than 0.25 second.

The influence may be effected both binocularly (myopia, hyperopia, prevention of vision fatigue) and monocularly (anisometropia, amblyopia), depending on the nature of the vision disorder and the dynamics of vision improvement. The duration of sessions and their number is determined on an individual basis, depending on the degree of vision fatigue and the dynamics of vision improvement.

It is also advisable that the range and discreteness of measuring the color parameters and brightness relations of the visual stimuli 1 and their color backgrounds 2, the distance to the visual stimuli 1, the degree of contrast between the visual stimulus 1 and its color background 2, the orientation and the angular dimensions of the visual stimuli 1 should be all selected so as both clearly and not clearly visible different visual stimuli 1, which have different angular dimensions (but not less than 0.5 angular minute), are available for perception, which is conditioned by a mean value of the limiting resolution of the human eye.

At all variants of influence it is necessary to be aimed at reaching the maximum vision definition of the least perceived visual stimuli 1. Systematic stimulation contributes to vision improvement, lowers the eye fatigability at long visual loads.

For effecting psychoemotional stimulation the visual stimuli 1 may be objects of the outward things, texts from books, motion pictures, video records, photo materials, images on video monitors and projection screens. The color-brightness characteristics of the visual stimuli 1 and their color backgrounds 2 may be modified by software and hardware means; printing and art means; with the use of transparent color polymeric, glass or liquid-crystal filters; by projection, computer, movie, photo and video means; as well as by sources of light of a given spectral range. However, not any modification of the color-brightness characteristics of the visual stimuli 1 and their own color backgrounds 2 results in the implementation of the claimed method. A modification should result in a change in the wavelength of the position of the center of intensity spectral distribution of the more intensive light flux out of the ones that form an image of the visual stimulus 1 and its own color background 2 on the retina. For example, changing the color of letters of a text printed on white paper will not result in achieving the objective of the invention. And a text, which is made in letters of different colors on black paper, enables to change the degree of accommodation tension of the eye in the course of reading it.

For the purpose of achieving a stable improvement of vision and maintaining it within the physiological norm it is advisable to conduct 1 or 2 half-hourly sessions daily.

The claimed method and various technical means have been applied for influencing the visual system of the eye at different disorders of the visual function.

### EXAMPLE 1

Schoolboy K. Age—11 years. Weak myopia. Prior to the vision improving sessions the vision acuity was OD=0.5 and OS=0.5; amplitude of relative accommodation 4.0 diopters; reserve of relative accommodation 3.0 diopters. During the period of 15 days for half an hour twice a day he was in training with colored text tables made in the form of a carrier with different color-brightness characteristics of its surface parts. After the conducted vision improving sessions the vision acuity is OD=0.8 and OS=0.8; amplitude of relative accommodation 7.0 diopters, reserve of relative accommodation 5.5 diopters.

### EXAMPLE 2

Schoolboy M. Age—10 years. Weak myopia, spasm of accommodation. Prior to the vision improving sessions the vision acuity was OD=0.3 and OS=0.5; amplitude of relative accommodation 2.5 diopters; reserve of relative accommodation 2.0 diopters. During the period of 15 days for half an hour twice a day he was in training with colored text tables and read with color filters laid on pages of a book. After the conducted vision improving sessions the vision acuity is OD=0.7 and OS=0.8; amplitude of relative accommodation 8.5 diopters, reserve of relative accommodation 5.5 diopters.

### EXAMPLE 3

Schoolgirl A. Age—13 years. Weak myopia, spasm of accommodation. Prior to the vision improving sessions the vision acuity was OD=0.6 and OS=0.5; amplitude of relative accommodation 3.5 diopters; reserve of relative accommodation 2.0 diopters. During the period of two months she daily prepared her homework, wearing glasses with replaceable color filters. After the conducted vision improving sessions the vision acuity is OD=1.0 and OS=0.9; amplitude of relative accommodation 7.5 diopters, reserve of relative accommodation 5.5 diopters.

### EXAMPLE 4

Teacher Yu. Age—48 years. Presbyopia, asthenopia. Prior to the vision improving sessions the vision acuity was OD=1.0 and OS=1.0; amplitude of relative accommodation 3.5 diopters; reserve of relative accommodation 1.0 diopter. For half a year he worn +0.5D glasses for close sight. For the period of two months he read newspaper texts, wearing glasses with replaceable color filters for half an hour daily. Also, when working with Microsoft Word 97 he changed the text background color every 2-3 minutes. After the conducted vision improving sessions the vision acuity is OD=1.5 and OS=1.5; amplitude of relative accommodation 6.5 diopters, reserve of relative accommodation 3.0 diopters. He quitted using glasses.

### EXAMPLE 5

Student K. Age—19 years. Asthenopia, weak spasm of accommodation. Passed a preventive course. Prior to the vision improving sessions the vision acuity was OD=0.9 and OS=0.9; amplitude of relative accommodation 5.0 diopters; reserve of relative accommodation 3.5 diopters. During the period of one month he prepared his homework, wearing glasses with replaceable color filters (approximately for two hours daily). After the preventive course the vision acuity is OD=1.5 and OS=1.5; amplitude of relative accommodation 8.5 diopters, reserve of relative accommodation 5.5 diopters.

### INDUSTRIAL APPLICABILITY

The claimed variants of the method and the device for spectral optical reflex therapy for improving vision may be most successfully used when presenting various visual information in printing products, computer software and technical means for prevention purposes and for improving the visual function.

### REFERENCES

(1) E. S. Avetisov, “Blizorukost” (“Myopia”), Medicina, 1986, p.183.

(2) Russian Federation Patent No. 2110239, A 61 F 9/00, published 10.05.98.

1. A method of spectral optical reflex therapy for improving vision, including cyclic modification of the degree of eye accommodation tension, which is effected by modifying the spectral distribution of the more intensive out of the two light fluxes, where one of them forms images of visual stimuli and the second one forms color backgrounds of visual stimuli, the said spectral distribution being modified by cyclically increasing and decreasing the wavelength of the position of the spectral intensity distribution center of the more intensive light flux out of the said two light fluxes.

2. A method according to claim 1, characterized in that the spectral distribution is modified by cyclically increasing and decreasing the wavelength of the position of the spectral intensity distribution center by the maximum value of not less than 3 nanometers.

3. A method according to claim 1, characterized in that the spectral distribution of the more intensive out of the said light fluxes is modified with the frequency of not more than 4 Hz.

4. A method of spectral optical reflex therapy for improving vision, including cyclic modification of the degree of eye accommodation tension, which is effected by cyclically modifying the relation between the intensities of the light fluxes, where one of them forms images of visual stimuli and the second one forms color backgrounds of visual stimuli, alternatively making one of the light fluxes more intensive, the said light fluxes are formed with spectral distributions wherein the wavelengths of the positions of the spectral intensity distribution centers before and after the modification of the relation between the intensities of the light fluxes differ.

5. A method according to claim 4, characterized in that the wavelengths of the positions of the spectral intensity distribution centers of light fluxes before and after the modification of the relation between their intensities are selected so that the difference between them would be not less than 3 nanometers.

6. A method according to claim 4, characterized in that the relation between the intensities of the light fluxes is modified with the frequency of not more than 4 Hz.

7. A device for spectral optical reflex therapy for improving vision, comprising a means made with the possibility of cyclically modifying the color parameters of visual stimuli being brighter than their color backgrounds, where the wavelengths of the positions of the spectral intensity distribution centers of the light fluxes, which are formed by the brighter visual stimuli before and after modifying their color parameters, differ.

8. A device according to claim 7, characterized in that the said device is made with the possibility of modifying the color-brightness characteristics of visual stimuli and their color backgrounds made of light-reflecting means for presenting visual information.

9. A device according to claim 8, characterized in that the said device is made as a carrier, on which visual stimuli, different as to their color parameters, are arranged.

10. A device according to claim 8, characterized in that the said device is made as a carrier and a transparent color filter, as arranged before the carrier, on which visual stimuli, different as to their color parameters, are arranged.

11. A device according to claim 8, characterized in that the said device is made as a carrier, on which visual stimuli are arranged, and a transparent color filter arranged before it.

12. A device according to claim 11, characterized in that the transparent color filter is made with parts differing by their color parameters.

13. A device according to claim 8, characterized in that the said means is made as a projection screen.

14. A device according to claim 7, characterized in that the said means is made with the possibility of modifying the color-brightness characteristics of visual stimuli and their color backgrounds made of light-irradiating means for presenting visual information.

15. A device according to claim 14, characterized in that the said means is made as a computer display.

16. A device according to claim 14, characterized in that the said means is made as a television screen.

17. A device for spectral optical reflex therapy for improving vision, comprising a means made with the possibility of cyclically modifying the color parameters of color backgrounds being brighter than visual stimuli made thereon, where the wavelengths of the positions of the spectral intensity distribution centers of the light fluxes, which are formed by the brighter color backgrounds before and after modifying their color parameters, differ.

18. A device according to claim 17, characterized in that the said means is made with the possibility of modifying the color-brightness characteristics of visual stimuli and their color backgrounds made of light-reflecting means for presenting visual information.

19. A device according to claim 18, characterized in that the said means is made as a carrier with parts different as to their color parameters, and visual stimuli arranged on the said carrier.

20. A device according to claim 18, characterized in that the said means is made as a carrier with parts different as to their color parameters and a transparent color filter, as arranged before the carrier, on which visual stimuli are arranged.

21. A device according to claim 18, characterized in that the said means is made as a carrier, on which visual stimuli are arranged, and a transparent color filter arranged before it.

22. A device according to claim 21, characterized in that the transparent color filter is made with parts differing by their color parameters.

23. A device according to claim 18, characterized in that the said means is made as a projection screen.

24. A device according to claim 17, characterized in that the said means is made with the possibility of modifying the color-brightness characteristics of visual stimuli and their color backgrounds made of light-irradiating means for presenting visual information.

25. A device according to claim 24, characterized in that the said means is made as a computer display.

26. A device according to claim 24, characterized in that the said means is made as a television screen.

27. A device for spectral optical reflex therapy for improving vision, comprising a means made with the possibility of cyclically the brightness relations of visual stimuli and their color backgrounds, where the visual stimuli and their color backgrounds are alternatively formed brighter in relation to each other, the color parameters of the brighter visual stimuli or their color backgrounds before and after modifying the brightness relations are different, and the wavelengths of the positions of the spectral intensity distribution centers of the light fluxes, which are formed by the brighter visual stimuli or their color backgrounds before or after modifying their brightness relations differ too.

28. A device according to claim 27, characterized in that the said means is made with the possibility of modifying the color-brightness characteristics of visual stimuli and their color backgrounds made of light-reflecting means for presenting visual information.

29. A device according to claim 28, characterized in that the said means is made as a carrier with parts different as to their color parameters, that parts being the color backgrounds of the visual stimuli arranged on the carrier and made with different color-brightness characteristics.

30. A device according to claim 28, characterized in that the said means is made as a carrier with parts different as to their color parameters and a transparent color filter, as arranged before the carrier, on which visual stimuli are arranged, being made opaque and with different color-brightness characteristics.

31. A device according to claim 28, characterized in that the said means is made as a projection screen.

32. A device according to claim 27, characterized in that the said means is made with the possibility of modifying the color-brightness characteristics of visual stimuli and their color backgrounds made of light-irradiating means for presenting visual information.

33. A device according to claim 32, characterized in that the said means is made as a computer display.

34. A device according to claim 32, characterized in that the said means is made as a television screen.
